(19) United States
(12) Patent Application Publication
(10) Pub. No.: US 2008/0045875 A1
(43) Pub. Date: Feb. 21, 2008

(54) Protective covering for construction stilts

(76) Inventor: Jason Lamprey, Webster, MA (US)

Correspondence Address: Law Office of Brett N. Dorny, Suite 12A, 386 West Main Street, Northborough, MA, 01532, US

(21) Appl. No.: 11/506,093

(22) Filed: Aug. 17, 2006

(51) Int. Cl. A61F 5/00; A61F 13/06

(52) U.S. Cl. 602/62; 602/3

(57) ABSTRACT

A protective covering for a plastering or other construction stilt is disclosed. The protective covering is formed as a cylindrical bag which attaches to the foot of the stilt at the bottom and to the calf of the wearer at the top. The protective covering prevents plaster, plaster dust, or other construction debris form contacting the stilt. The protective covering may also be attached to the base of the stilt. A flexible fabric material may be used for the protective covering.

### BACKGROUND OF THE INVENTION

1. Field of the Invention

The present invention relates to protective coverings. More specifically, it relates to a protective covering for stilts for use in plastering or other construction trades.

2. Discussion of Related Art

Plasterers commonly wear stilts in order to reach the ceilings and high parts of walls while performing their services. The stilts are strapped to the legs of the user so that the person can easily reach high locations without the need to climb up and down a ladder. In order to provide proper balance and maximum support, the stilts include a complicated structure made up of multiple parts. However, plastering can be a messy project. Plaster can be dropped and plaster dust gets in the air. Therefore, the stilts commonly get coated with plaster debris. This causes the stilts to deteriorate and fail. Since stilts are expensive, a major cost to the plasterer is replacing stilts which have failed due to plaster and plaster debris on the stilts. Therefore, a need exists for a device to prevent plaster and plaster debris from coming into contact with the stilts during the plastering process. Similarly, stilts are used for other construction trades, including insulators, painters, board hangers, and drywall tapers. These trades also generate debris which adheres to and damages stilts. Therefore, a need exists for a device to prevent damage to stilts from any construction activity.

### SUMMARY OF THE INVENTION

The present invention provides a structure for protecting stilts used in plastering and other construction trades. According to one aspect of the invention, the structure includes a protective covering over the stilts and feet of the wearer. According to another aspect of the invention, the protective covering up is made of a fabric material. According to another aspect of the invention, the protective covering is formed as a bag which is placed over the top of the stilts. According to another aspect and invention, the protective covering includes an attachment at the bottom of the covering to connect to the stilts to prevent plaster and plaster debris from entering within the protective covering. According to another aspect of invention, the protective covering includes a portion which extends across a leg of the wearer.

### BRIEF DESCRIPTION OF THE DRAWINGS

FIG. 1 is a front view of a protective covering according to an embodiment of invention.

FIG. 2 is a cross sectional view of the protective covering of FIG. 1 on a plastering stilt.

### DETAILED DESCRIPTION

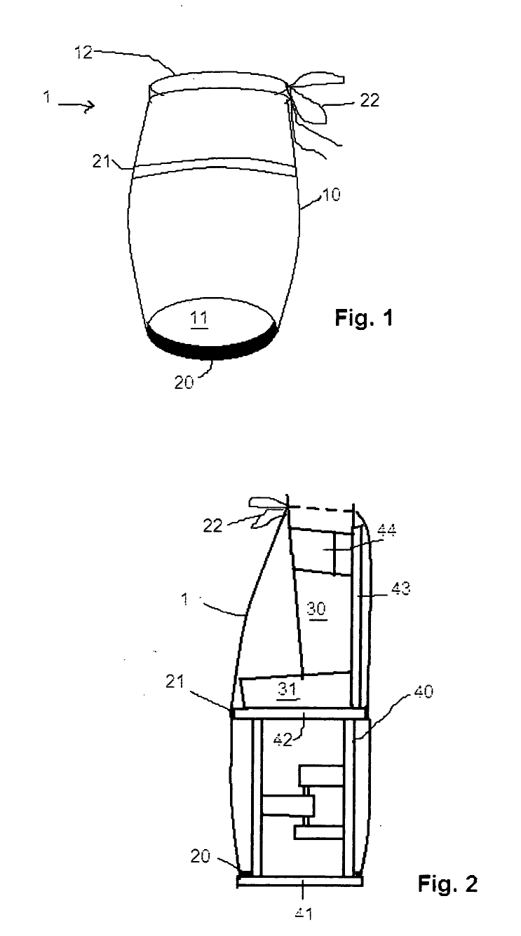

The present invention provides a protective covering for plastering stilts. As illustrated in FIG. 1, the protective covering 1 is formed as a cylindrical bag 10 having upper 12 and lower 11 openings. The lower opening 11 has elastic 20 along the edge. The upper opening 12 has a drawstring 22. Velcro 21 is also positioned on the interior of the bag 10 at an intermediate portion. FIG. 2 illustrates the arrangement between the protective covering 1 and a plastering stilt 40. As is known in the art, a stilt 40 includes a foot 41, a spaced apart base 42, a support 43 and a strap 44. The wearer places his foot 31 on the base 42 so that the leg 30 extends along the support 43. The strap 44 connects the support 43 to the calf of the wearer. Other straps may be included to connect to the foot or ankle as well as the calf. As is illustrated in FIG. 2, the protective covering 1 is positioned around the stilt 40. The elastic 20 maintains the lower opening around the stilt 40 near the foot 41. Alternatively, the elastic 20 could be replaced with Velcro. Velcro could be attached to the foot 41 of the stilt 40 to connect with the lower opening of the protective covering 1. The drawstring 22 at the upper opening 12 tightens the protective covering 1 around the calf of the wearer above the strap 44. In this manner the entire stilt 40, except for a lower portion of the foot 41, is enclosed within the protective covering 1. Velcro may also be attached to the base 42 of the stilt. This allows the Velcro 21 at the intermediate portion of the protective covering 1 to be attached to the base. When used in this manner, the wearer can remove the stilt 40 while a lower portion of the stilt 40 remains covered by the protective covering 1. After attaching the stilt 40 to his or her leg, the wearer lifts an upper portion of the protective covering 1 over the top part of the stilt 40 and tightens the drawstring 22.

Preferably, the protective covering 1 his formed of a closely woven fabric to prevent the plaster and debris from passing through the protective covering 1. The protective covering 1 may also be treated with a plastic or other material on the exterior or interior surface in order to preclude any entry of plaster dust. Of course, other materials, such as polymers and plastics, which are sufficiently flexible for use of the stilts may be used for the protective covering of the present invention.

The protective covering 1 has been described as using Velcro 20, 21 and a drawstring 22 for attaching it to the stilt and leg of the wearer. Other mechanisms may also be used for attaching the protective covering. For example, the lower opening 11 may also use a drawstring, which is tightened around the lower portion of the stilt. Adhesives may also be used to attach the protective covering 1 to the foot 41 and base 42 of the stilt 40.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A protective covering for a stilt comprising:
a cylinder of a flexible material having a lower opening and an upper opening, the cylinder extending so as to enclose portions of the stilt above a foot of the stilt, the lower opening being positioned near the foot of the stilt and the upper opening being positioned above the stilt;
means for retaining the lower opening of the cylinder of flexible material near the foot of the stilt; and
means for retaining the upper opening around a calf of a wearer of the stilt.

2. The protective covering for a stilt according to claim 1, wherein the means for retaining the lower opening includes an elastic.

3. The protective covering for a stilt according to claim 1, wherein the means for retaining the upper opening includes a drawstring.

4. The protective covering for a stilt according to claim 1, wherein the means for retaining the lower opening includes a hook and loop fastener.

5. The protective covering for a stilt according to claim 1, wherein the flexible material includes a fabric.

6. The protective covering for a stilt according to claim 1, further comprising:
means, around a central portion of the cylinder of flexible material between the lower opening and upper opening, for connecting the cylinder of flexible material to a base of the stilt.

7. The protective covering for a stilt according to claim 6, wherein the means for connecting the cylinder of flexible material includes a hook and loop fastener.

8. The protective covering for a stilt according to claim 4, wherein the hook and loop fastener includes:
a first part attachable to the stilt; and
a second part, which mates with the first part, attached to the lower opening.

9. The protective covering for a stilt according to claim 7, wherein the hook and loop fastener includes:
a first part attachable to the base of the stilt; and
a second part, which mates with the first part, attached to the central portion of the cylinder of flexible material.
